(19) United States
(12) Patent Application Publication
(10) Pub. No.: US 2026/0207727 A1
(43) Pub. Date: Jul. 23, 2026

(54) Mutated factor H binding proteins (fHbp) of Neisseria meningitidis, their compositions and use thereof

(71) Applicants: TECHINVENTION LIFECARE PRIVATE LIMITED, Mumbai (IN); BIOGRIT TECHNOLOGIES INC, Toronto (CA)

(72) Inventors: Sarmad HANIF, Mumbai (IN); Syed S AHMED, Mumbai (IN)

(73) Assignees: TECHINVENTION LIFECARE PRIVATE LIMITED, Mumbai (IN); BIOGRIT TECHNOLOGIES INC, Toronto, ON (CA)

(21) Appl. No.: 19/134,226

(22) PCT Filed: Nov. 3, 2023
(86) PCT No.: PCT/IN2023/051008

§ 371 Date: May 30, 2025

(30) Foreign Application Priority Data
Nov. 4, 2022 (IN) 202221063189

(51) Int. Cl. A61K 39/095; A61K 9/00; A61K 38/00; A61K 39/00; A61K 39/385; A61P 31/04; C07K 14/22

(52) U.S. Cl. CPC A61K 39/095; A61K 9/0019; A61K 39/385; A61P 31/04; C07K 14/22; A61K 38/00; A61K 2039/54; A61K 2039/575; A61K 2039/6087; C07K 2319/02

(57) ABSTRACT

The present invention relates to Bio-pharmaceuticals. Specifically, the present invention relates to mutated Factor H binding proteins (fHbp) of N. meningitidis serogroup B. Further, the present invention relates to compositions of factor H binding protein (fHbp) for the prevention of N. meningitidis. The present invention particularly relates to mutated factor H binding proteins (fHbp) of N. meningitidis, their compositions and use thereof. The present invention further relates to the use of said mutated Factor H binding proteins (fHbp) in the treatment or prevention or diagnosis of N. meningitidis disease by at least one of serogroup A, C, B, Y and W disease. The present invention also relates to testing of factor H binding proteins (fHbp) of N. meningitidis serogroup B composition in animal models to determine its efficacy. The invention specifically relates to a pharmaceutical composition comprising said mutated factor H binding proteins (fHbp) for use in the treatment or prevention or diagnosis of N. meningitidis serogroup B disease.

### FIELD OF THE INVENTION

The present invention relates to Bio-pharmaceuticals. Specifically, the present invention relates to mutated factor H binding proteins (fHbp) of Neisseria meningitidis. Further, the present invention relates to compositions of mutated factor H binding protein (fHbp) for the prevention of N. meningitidis. The present invention particularly relates to the mutated factor H binding proteins (fHbp) of N. meningitidis, their compositions and uses thereof. The present invention further relates to the use of said mutated factor H binding proteins (fHbp) in the treatment or prevention or diagnosis of N. meningitidis disease by at least one of serogroup A, C, B, Y, X and W disease. The invention specifically relates to a pharmaceutical composition comprising said mutated factor H binding proteins (fHbp) for use in the treatment or prevention or diagnosis of N. meningitidis serogroup B disease. The invention specifically relates to a pharmaceutical composition comprising said mutated factor H binding proteins (fHbp) for use as prophylactic or therapeutic Vaccine.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

N. meningitidis (Men) is a Gram-negative encapsulated bacteria which can cause disease such as sepsis, meningitis and death. It has 13 serogroups with the most prevalent (serogroups) as A, B, C, W, Y and X (MenA, MenB, MenC, MenW, MenY, and MenX) [McNeil et al., Vaccine; 27, 2009, 3417-3421; Tapia et al., N Engl J Med; 2021 Jun. 3; 384(22): 2115-2123]. MenB is prevalent around the globe [Garcia et al., npj Vaccines 6, 130(2021)].

Polysaccharide (PS) capsule present in the bacteria is considered as an important virulence factor and has been successfully used for the development of PS based conjugate vaccines for MenA, MenC, MenW, MenY and also for MenX, which is in development. However, MenB PS capsule is poorly immunogenic in humans and also resembles the human neuronal cells [Goldblatt, Clin Exp Immunol. 2000; 119:1-3; Luo et al., AAPS J. 2016 November; 18(6): 1562-1575; Tapia et al,., N Engl J Med; 2021 Jun. 3; 384(22): 2115-2123; McNeil et al., Vaccine; 27, 2009, 3417-3421]. However there are other various (surface) virulence factors of N. meningitidis [Caesar et al., Microbial Pathogenesis, 57, 2013, 33-40].

fHbp (an outer membrane native lipoprotein of approximately 30-32 kDa in size) has proved to be a virulence factor for N. meningitidis and a target for functional bactericidal antibodies. fHbp is also known as lipoprotein 2086 (LP2086) and genome-derived Neisseria antigen (GNA 1870), fHbp binds to the human factor H (fH), and its binding allows the bacterium to resist complement-mediated killing and evade the immune invasion [Yee W X, Barnes G et al., Trends Microbiol. 2023 August; 31(8): 805-815. Abad et al., J Infect. 2021 April; 82(4): 37-44; Fletcher et al., Infect Immun. 2004 April; 72(4): 2088-100; Gandhi et al., Postgrad Med. 2016 August; 128(6): 548-56; McNeil et al., Microbiol Mol Biol Rev. 2013 June; 77(2): 234-52].

Sequencing of the fHbp gene in diverse MenB strains revealed the presence of three main variants (var1, var2, var3) [Masignani et.al., J Exp Med. 2003 Mar. 17; 197(6): 789-99; Brehony et al., N. Microbiology (Reading). 2009 December; 155 (Pt12): 4155-4169;], or two subfamilies (A and B: corresponding to variants 2/3 and 1, respectively) [Gandhi et al., Postgrad Med. 2016 August; 128(6): 548-56].

TABLE 1
fHbp Classification

| Subfamilies | Variants |
|---|---|
| (Pfizer Designation) | (Novartis Designation) |
| A | V.2 |
| | V.3 |
| B | V.1 |

Trumenba® by Pfizer and Bexsero® by GSK are the two licensed fHbp containing vaccines. Although both have fHbp's, however the composition of both of them is unlike. Trumenba® is a bivalent fHbp lipoprotein based vaccine which is recombinantly produced, it has two fHbp's one each from subfamily A (A05) and subfamily B (B01) [Gandhi et al., Postgrad Med. 2016 August; 128(6): 548-56; Feavers and Maiden, Clin Vaccine Immunol. 2017 May 5; 24(5): e00566-16].

Bexsero® also called as 4CMenB has four antigens as Neisserial adhesin A (NadA), Neisserial heparin-binding antigen (NHBA) fused to protein GNA1030, fHbp (non lipidated, subfamily B and variant B24) fused with protein GNA2091 and an Outer Membrane Vesicle (OMV) [Gandhi et al., Postgrad Med. 2016 August; 128(6): 548-56; Feavers and Maiden, Clin Vaccine Immunol. 2017 May 5; 24(5): e00566-16].

Meningococcal vaccine efficacy can be measured by Serum Bactericidal Assay (SBA) (a correlate of protection) and is considered as a gold standard correlate of immunity [Borrow et al., J Infect. 2020 December; 81(6): 862-872; Dretler et al., Hum Vaccin Immunother. 2018 May 4; 14(5): 1146-1160]. It has been demonstrated preclinically that fHBP-based vaccines elicited serum bactericidal antibodies and were capable of killing MenB strains in SBA's. Preclinical findings led to the advancement fHBP based vaccines into clinical trials and showed that it could produce bactericidal antibodies in human subjects as well [Caesar et al., Microbial Pathogenesis, 57, 2013, 33-40; McNeil et al., Microbiol Mol Biol Rev. 2013 June; 77(2): 234-52]. There remains an unmet need for a broader fHbp polypeptide that can elicit effective antigenic responses.

For the present invention A02, A42, A46, B44 and B107 of the fHbp family have been selected for mutations in their amino acid sequence and to produce them as recombinant proteins disclosed herein.

The advantage for the inclusion of different subfamily fHbp antigens is for the broader coverage. It is observed, that for broader coverage, fHbp from both subfamilies (A and B) are required as SBA killing against the subfamily B isolate was not observed when subfamily A serum was used [McNeil et al., Microbiol Mol Biol Rev. 2013 June; 77(2): 234-52]. Therefore it is desirable and judicious to use fHbp's from both the subfamilies for broader coverage.

The development of N. meningitidis serogroup B vaccine will plausibly complement the unmet need of meningococcal B disease by having coverage rate in the populations globally [https://www.who.int/es/publications/i/item/9789240026407 accessed on Nov. 1, 2023; Safadi et al., Expert Rev Vaccines. 2021 April; 20(4): 401-414]

Although there are different companies working on N. meningitidis, there is still a need to explore the novel ways and compositions of N. meningitidis serogroup B vaccine that could be more economical, easily manufactured and scalable and can overcome deficiencies associated with the known arts.

The present invention provides a novel, specific and industrially advantageous mutated factor H binding proteins (fHbp) of N. meningitidis. The aim of the present invention is to perform modifications/mutation in the amino acid (aa) sequences of both subfamily A and B fHbp proteins. These modifications shall be antigenic and therefore having the capability to elicit the desired immune response to prevent N. meningitidis serogroup B disease. The inclusion of fHbp proteins from both subfamily A and subfamily B is preferred to provide broader coverage. This Vaccine shall be fulfilling the unmet medical need for N. meningitidis prophylaxis.

### OBJECTS OF THE INVENTION

An object of the present invention is to provide mutated factor H binding proteins (fHbp) of N. meningitidis.

An object of the present invention is to provide mutated factor H binding proteins (fHbp) of A02, A42, A46, B44 and B107 of N. meningitidis.

An object of the present invention is to provide mutated factor H binding proteins (fHbp) of A02, A42, A46, B44 and B107 of N. meningitidis family selected from sequence ID nos. 1, 2, 3, 4 and 5 respectively.

An object of the present invention is to provide a pharmaceutical composition comprising at least two of the following:
a) mutated protein of fHbp family A02 of N. meningitidis (protein of sequence ID. 1);
b) mutated protein of fHbp family A42 of N. meningitidis (protein of sequence ID No. 2);
c) mutated protein of fHbp family A46 of N. meningitidis (protein of sequence ID No. 3);
d) mutated protein of fHbp family B44 of N. meningitidis (protein of sequence ID No. 4); and
e) mutated protein of fHbp family B107 of N. meningitidis (protein of sequence ID No. 5).

An object of the present invention is to provide a pharmaceutical composition comprising at least two of the following:
a) mutated protein of fHbp family A02 of N. meningitidis (at least 90% sequence identity to sequence ID No. 1);
b) mutated protein of fHbp family A42 of N. meningitidis (at least 90% sequence identity to sequence ID No. 2);
c) mutated protein of fHbp family A46 of N. meningitidis (at least 90% sequence identity to sequence ID No. 3);
d) mutated protein of fHbp family B44 of N. meningitidis (at least 90% sequence identity to sequence ID No. 4); and
e) mutated protein of fHbp family B107 of N. meningitidis (at least 90% sequence identity to sequence ID No. 5).

Another object of the present invention is to provide a pharmaceutical composition as described herein above for prophylaxis against meningococcal diseases caused by pathogenic N. meningitidis.

Another objective of the present invention is to provide a pharmaceutical composition as described herein above for prophylaxis against meningococcal diseases caused by all six prevalent serogroups (MenA, MenB, MenC, MenW, MenY, and MenX) of N. meningitidis which are responsible for invasive meningitis and septicemia.

Another objective of the present invention is to provide a pharmaceutical composition as described herein above for prophylaxis against meningococcal diseases caused by type B antigenic serogroup of N. meningitidis.

Another objective of the present invention is to provide a pharmaceutical composition as described herein above use as prophylactic or therapeutic Vaccine.

Another objective of the present invention is to provide a pharmaceutical composition as described herein above for use as prophylactic or therapeutic Vaccine against meningococcal diseases caused by type B antigenic serogroup of N. meningitidis.

Still another object of the present invention is to provide a process for preparation of mutated factor H binding proteins (fHbp) of A02, A42, A46, B44 and B107 of N. meningitidis.

Another object of the present invention is to provide a process for preparation of mutated factor H binding proteins (fHbp) of A02, A42, A46, B44 and B107 of N. meningitidis selected from recombinant process, synthetic process or combination process thereof.

Another object of the present invention is to provide a process for preparation of a pharmaceutical composition comprising at least two of the following:
a) mutated protein of fHbp family A02 of N. meningitidis (at least 90% sequence identity to sequence ID No. 1);
b) mutated protein of fHbp family A42 of N. meningitidis (at least 90% sequence identity to sequence ID No. 2);
c) mutated protein of fHbp family A46 of N. meningitidis (at least 90% sequence identity to sequence ID No. 3);
d) mutated protein of fHbp family B44 of N. meningitidis (at least 90% sequence identity to sequence ID No. 4); and
e) mutated protein of fHbp family B107 of N. meningitidis (at least 90% sequence identity to sequence ID No. 5).

### SUMMARY OF THE INVENTION

This summary is provided to introduce a selection of concepts in a simplified form that are further described below in Detailed Description section. This summary is not intended to identify key features or essential features of the claimed subject matter, nor is it intended to be used as an aid in determining the scope of the claimed subject matter.

The present invention relates to mutated factor H binding proteins (fHbp) of N. meningitidis.

In one aspect, the present invention relates to mutated factor H binding proteins (fHbp) of A02, A42, A46, B44 and B107 of N. meningitidis serogroup B.

In another aspect, the present invention relates to a mutated factor H binding proteins (fHbp) of A02, A42, A46, B44 and B107 of N. meningitidis serogroup B as sequence ID nos. 1, 2, 3, 4 and 5 respectively.

In one aspect, the present invention relates to a pharmaceutical composition comprising at least two of the following:
a) mutated protein of fHbp family A02 of N. meningitidis (protein of sequence ID. 1);
b) mutated protein of fHbp family A42 of N. meningitidis (protein of sequence ID. 2);
c) mutated protein of fHbp family A46 of N. meningitidis (protein of sequence ID. 3);
d) mutated protein of fHbp family B44 of N. meningitidis (protein of sequence ID. 4); and
e) mutated protein of fHbp family B107 of N. meningitidis (protein of sequence ID. 5).

In yet another aspect, the present invention relates to a pharmaceutical composition as described herein above for prophylaxis against meningococcal diseases caused by pathogenic N. meningitidis.

In yet another aspect, the present invention relates to a pharmaceutical composition as described herein above for use as prophylactic or therapeutic Vaccine.

In yet another aspect, the present invention relates to a pharmaceutical composition as described herein above for use as prophylactic or therapeutic Vaccine against meningococcal diseases caused by type B antigenic serogroup of N. meningitidis.

In one aspect, the present invention relates to a pharmaceutical composition as described herein above for prophylaxis against meningococcal diseases caused by MenA, MenB, MenC, MenW, MenY, and MenX serogroups of N. meningitidis.

In one aspect, the present invention relates to a pharmaceutical composition as described herein above for prophylaxis against meningococcal diseases caused by type B antigenic serogroup of N. meningitidis which are responsible for invasive meningitis and septicaemia.

In still another aspect, the present invention relates to a process for preparation of mutated factor H binding proteins (fHbp) of A02, A42, A46, B44 and B107 of N. meningitidis.

In another aspect, the present invention relates to a process for preparation of mutated factor H binding proteins (fHbp) of A02, A42, A46, B44 and B107 of N. meningitidis selected from recombinant process, synthetic process or combination process thereof.

### DRAWINGS

The following drawings form part of the present specification and are included to further illustrate aspects of the present disclosure. The disclosure may be better understood by reference to the drawings in combination with the detailed description of the specific embodiments presented herein.

FIG. 1 is SDS PAGE showing induction of mutated subfamily B fHbp Sequence ID 5.

FIG. 2 is SDS PAGE showing induction of mutated subfamily B fHbp Sequence ID 4.

FIG. 3 is SDS PAGE showing induction of mutated subfamily A fHbp Sequence ID 2.

FIG. 4 is SDS PAGE showing induction of mutated subfamily A fHbp Sequence ID 1 and Sequence ID 3.

FIG. 5 is SDS PAGE showing image of purified Sequence ID 3 and Sequence ID 5.

FIG. 6 shows Western blot image of purified Sequence ID 3 and Sequence ID 5.

FIG. 7 shows graph of IgG response against the mutated subfamily A fHbp

FIG. 8 shows graph of IgG response against the mutated subfamily B fHbp

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise.

Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any unclaimed element essential to the practice of the invention.

The description that follows, and the embodiments described therein, is provided by way of illustration of an example, or examples, of particular embodiments of the principles and aspects of the present disclosure. These examples are provided for the purposes of explanation, and not of limitation, of those principles and of the disclosure.

The headings and abstract of the invention provided herein are for convenience only and do not interpret the scope or meaning of the embodiments.

Various terms as used herein are shown below. To the extent a term used in a claim is not defined below, it should be given the broadest definition persons in the pertinent art have given that term as reflected in printed publications and issued patents at the time of filing.

The term “protein” according to the present invention means, but not limited to, refers to large biomolecules and macromolecules that comprise one or more long chains of amino acid residues. The chain amino acid residue may be of any length and may be linear or branched. This term encompasses all the naturally occurring or modified/recombinant proteins.

The term “immunological composition” according to the present invention means, but not limited to, refers to composition which elicit an immune response. The term immunological composition is interchangeably used with other term like “composition”, “pharmaceutical composition”, “vaccine composition” or “vaccine formulation” which has same meaning as defined for immunological composition

The term “Neisseria Meningitidis” or “N. meningitidis” according to the present invention means, but not limited to, refers to diplococcal, Gram-negative, and human commensal bacterium of the upper respiratory tract. The pathogen can invade the mucosa and gain access to the bloodstream, resulting meningitis, severe sepsis, or localized infections in joints and heart.

The term “mutated factor H binding proteins (fHbp)” according to present invention means, but not limited to, altered or modified versions of the factor H binding proteins. These mutated fHbp proteins have been manipulated to possess changes or mutations in their amino acid sequences.

The term “Adjuvant/adjuvant component” according to the present invention means, but not limited to, an adjuvant or an adjuvant component in the broadest sense is typically a (e.g., pharmacological or immunological) agent or composition that may modify, e.g., enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of anti-gens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immuno potentiators, antigenic delivery systems or even combinations thereof. The term “adjuvant” is typically understood not to comprise agents which confer immunity by themselves. An adjuvant assists the immune system unspecifically to enhance the antigen-specific immune response by e.g., promoting presentation of an antigen to the immune system or induction of an unspecific innate immune response. Furthermore, an adjuvant may preferably e.g., modulate the antigen-specific immune response by e.g., shifting the dominating Th2-based antigen specific response to a more Th1-based antigen specific response or vice versa and/or by inducing of mucosal immune responses and/or increased IgA titers. Accordingly, an adjuvant may favourably modulate cytokine ex-pression/secretion, antigen presentation, type of immune response etc.

Trehalose or D-glucopyranosyl alpha-D-glucopyranoside is a disaccharide known for its protective action on proteins when they are subjected to high temperatures, in particular during drying or lyophilization operations. According to the teaching of document U.S. Pat. No. 4,891,319, its protective action could be explained by a substitution of water molecules by molecules of trehalose, the 2 compounds comprising OH functions. Trehalose is also known in the prior art as a cell protector.

Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccines. These may be co-administered by any route, e.g., intramuscularly, subcutaneous, IV or intradermal injections.

The term “therapeutically effective amount” according to the present invention means, but not limited to, refers to an amount of the active ingredient (i.e., therapeutic protein or antibody) sufficient to produce the desired therapeutic effect in a human or animal, e.g., the amount necessary to treat, cure, prevent, or inhibit development and progression of disease or the symptoms thereof and/or the amount necessary to ameliorate symptoms or cause regression of disease. Such a therapeutically effective amount may vary depending on the structure and potency of the active ingredient and the contemplated mode of administration.

The term “treatment” according to the present invention means, but not limited to, refers to both therapeutic treatment and prophylactic or preventative measures. Those in need of treatment include those individuals, such as humans and animals, already with the disorder or condition to be treated as well as those prone to have the disorder or those in which the disorder is to be prevented. As used herein, “treatment” also includes reduction of the likelihood of obtaining the disorder, reduction of the severity of the disorder in those already afflicted, and the induction of regression of the disorder or symptoms thereof.

The term “pharmaceutically-acceptable carrier” according to the present invention means, but not limited to, refers to a liquid filler, diluent or encapsulating substance that may be safely used in systemic administration. Depending upon the particular route of administration, a variety of pharmaceutically acceptable carriers, well known in the art may be used. These carriers may be selected from a group including sugars, starches, cellulose and its derivatives, malt, gelatin, talc, calcium sulfate, vegetable oils, synthetic oils, polyols, alginic acid, phosphate buffered solutions including phosphate buffered saline, emulsifiers, isotonic saline, and pyrogen free water. In particular, pharmaceutically acceptable carriers may contain different components such as a buffer, sterile water for injection, normal saline or phosphate-buffered saline, sucrose, histidine, salts and polysorbate. Terms such as “physiologically acceptable”, “diluent” or “excipient” can be used interchangeably.

Mutated fHbp:

The present invention provides mutated factor H binding proteins (fHbp) of N. meningitidis.

The present invention provides mutated factor H binding proteins (fHbp) of N. meningitidis serogroup B.

In one embodiments, the present invention provides mutated factor H binding proteins (fHbp) of A02, A42, A46, B44 and B107 of N. meningitidis family.

In one embodiments, the present invention provides a mutated factor H binding proteins (fHbp) of A02, A42, A46, B44 and B107 of N. meningitidis fHbp family having at least 90% sequence identity to sequence ID nos. 1, 2, 3, 4 and 5 respectively.

In further embodiment, the present invention provides mutated factor H binding proteins (fHbp) of A02, A42, A46, B44 and B107 of N. meningitidis family as a medicament or active pharmaceutical ingredient or drug substance. Moreover, the present invention provides a pharmaceutical composition of the invention for use in the prevention of the infection of the N. meningitidis.

Process of Preparation of Mutated fHbp:

In one embodiments, the present invention provides a process for preparation of mutated factor H binding proteins (fHbp) of A02, A42, A46, B44 and B107 of N. meningitidis.

In one embodiments, the present invention provides a process of preparation of mutated factor H binding proteins (fHbp) of A02, A42, A46, B44 and B107 of N. meningitidis having at least 90% sequence identity to sequence ID nos. 1, 2, 3, 4 and 5 respectively.

In another embodiment, the present invention provides a process a process for preparation of mutated factor H binding proteins (fHbp) of A02, A42, A46, B44 and B107 of N. meningitidis selected from recombinant process, synthetic process or combination of both thereof.

Still another embodiment of the present invention provides a process of preparation of mutated factor H binding proteins (fHbp) of A02, A42, A46, B44 and B107 of N. meningitidis serogroup B fHbp comprising steps of:
a) Cloning of the individual fHbp gene with the signal sequence into expression vector,
b) Plasmid extraction,
c) Transformation
d) Induction of the respective fHbp's
e) Purification of respective fHbp's

In yet another embodiment of the present invention provides a process of preparation of mutated factor H binding proteins (fHbp) of A02, A42, A46, B44 and B107 of N. meningitidis serogroup B fHbp wherein the cloning is done in pET303 vector.

In yet another embodiment of the present invention provides a process of preparation of mutated factor H binding proteins (fHbp) of A02, A42, A46, B44 and B107 of N. meningitidis serogroup B fHbp wherein the step (a) of cloning also involve codon optimization, gene synthesis, sub cloning, transformation.

In yet another embodiment of the present invention provide a process of preparation of mutated factor H binding proteins (fHbp) of A02, A42, A46, B44 and B107 of N. meningitidis serogroup B fHbp wherein the step (e) of purification is carried out using method selected from, but not limited to, Affinity Chromatography, Ion Exchange Chromatography, Size Exclusion Chromatography (SEC), Hydrophobic Interaction Chromatography (HIC), Immunoaffinity Chromatography, His-Tag Affinity Chromatography, Protein A/G Purification, Precipitation Methods, Two-Phase Partitioning, Ultrafiltration, Differential Solubility.

### Composition:

In one embodiment, the present invention provides a pharmaceutical composition comprising at least two of the following:
a) mutated protein of fHbp family A02 of N. meningitidis (protein of sequence ID. 1);
b) mutated protein of fHbp family A42 of N. meningitidis (protein of sequence ID. 2);
c) mutated protein of fHbp family A46 of N. meningitidis (protein of sequence ID. 3);
d) mutated protein of fHbp family B44 of N. meningitidis (protein of sequence ID. 4); and
e) mutated protein of fHbp family B107 of N. meningitidis (protein of sequence ID. 5).

In still another embodiment, the present invention provides a pharmaceutical composition comprising at least two of the following:
a) mutated protein of fHbp family A02 of N. meningitidis (at least 90% sequence identity to sequence ID No. 1);
b) mutated protein of fHbp family A42 of N. meningitidis (at least 90% sequence identity to sequence ID No. 2);
c) mutated protein of fHbp family A46 of N. meningitidis (at least 90% sequence identity to sequence ID No. 3);
d) mutated protein of fHbp family B44 of N. meningitidis (at least 90% sequence identity to sequence ID No. 4); and
e) mutated protein of fHbp family B107 of N. meningitidis (at least 90% sequence identity to sequence ID No. 5).

In another embodiment, the present invention provides a method of production of a mutated factor H binding proteins (fHbp) of A02, A42, A46, B44 and B107 of N. meningitidis serogroup B fHbp having at least 90% sequence identity to sequence ID nos. 1, 2, 3, 4 and 5 respectively, said method comprises use of host cells selected from, but not limited to, cells of mammalian, plant, insect, fungal or bacterial origin. Host cells of the present invention include but are not limited to eukaryotic cells such as mammalian cells, e.g. hamster, rabbit, rat, pig, mouse, etc.; avian cells, e.g. duck, chicken, quail, etc.; insect cells or other animal cells; plant cells and fungal cells, e.g. corn, tobacco, Saccharomyces cerevisiae, Pichia pastoris; prokaryotic cells such as E. coli; and other cells used in the art for the production of monoclonal antibodies and other binding proteins.

### Composition/Formulation:

In one embodiment, the present invention provides a pharmaceutical composition comprising at least two of the following:
a) mutated protein of fHbp family A02 of N. meningitidis (at least 90% sequence identity to sequence ID No. 1);
b) mutated protein of fHbp family A42 of N. meningitidis (at least 90% sequence identity to sequence ID No. 2);
c) mutated protein of fHbp family A46 of N. meningitidis (at least 90% sequence identity to sequence ID No. 3);
d) mutated protein of fHbp family B44 of N. meningitidis (at least 90% sequence identity to sequence ID No. 4); and
e) mutated protein of fHbp family B107 of N. meningitidis (at least 90% sequence identity to sequence ID No. 5).

In one embodiment, the present invention provides a pharmaceutical composition comprising at least one of mutated factor H binding proteins (fHbp) of A02, A42, A46, B44 and B107 of N. meningitidis useful for the treatment or prevention or diagnosis of N. meningitidis disease caused by MenA, MenB, MenC, MenW, MenY, and MenX serogroups of N. meningitides.

In another embodiment, the present invention provides a pharmaceutical composition comprising at least one of mutated factor H binding proteins (fHbp) of A02, A42, A46, B44 and B107 of N. meningitidis serogroup B useful for the treatment or prevention or diagnosis of N. meningitidis disease.

In yet another embodiment, the present invention provides a pharmaceutical composition comprising at least one of mutated factor H binding proteins (fHbp) of A02, A42, A46, B44 and B107 of N. meningitidis of the invention further containing a pharmaceutically acceptable carrier, diluent or adjuvant.

In yet another embodiment, the present invention provides a pharmaceutical composition comprising at least one of mutated factor H binding proteins (fHbp) of A02, A42, A46, B44 and B107 of N. meningitidis of the invention further comprising pharmaceutically acceptable carrier selected from but not limited to, buffer, antioxidant, preservative, isotonic agent and chelating agent.

In yet another embodiment, the present invention provides a pharmaceutical composition comprising fully liquid and/or lyophilized modified fHbp from subfamily A and/or modified fHbp from subfamily B of N. meningitidis serogroup B and/or mixed with fully liquid and/or lyophilized conjugated capsular polysaccharides from N. meningitidis serogroup A, C, W135, X and/or Y.

### Process for Preparation of Composition/Formulation:

In one embodiment, the present invention provides a process of preparation of a pharmaceutical composition comprising at least two of the following:
a) mutated protein of fHbp family A02 of N. meningitidis (at least 90% sequence identity to sequence ID No. 1);
b) mutated protein of fHbp family A42 of N. meningitidis (at least 90% sequence identity to sequence ID No. 2);
c) mutated protein of fHbp family A46 of N. meningitidis (at least 90% sequence identity to sequence ID No. 3);
d) mutated protein of fHbp family B44 of N. meningitidis (at least 90% sequence identity to sequence ID No. 4); and
e) mutated protein of fHbp family B107 of N. meningitidis (at least 90% sequence identity to sequence ID No. 5).

### Diagnostics

In one of the embodiment, the present invention provides a diagnostic kit containing at least one of at least two of the following:
a) mutated protein of fHbp family A02 of N. meningitidis (at least 90% sequence identity to sequence ID No. 1);
b) mutated protein of fHbp family A42 of N. meningitidis (at least 90% sequence identity to sequence ID No. 2);
c) mutated protein of fHbp family A46 of N. meningitidis (at least 90% sequence identity to sequence ID No. 3);
d) mutated protein of fHbp family B44 of N. meningitidis (at least 90% sequence identity to sequence ID No. 4); and
e) mutated protein of fHbp family B107 of N. meningitidis (at least 90% sequence identity to sequence ID No. 5).

In one of the embodiment, the present invention provide a pharmaceutical composition of mutated factor H binding proteins (fHbp) of A02, A42, A46, B44 and B107 of N. meningitidis which is immunogenic.

In one of the embodiment, the present invention provide a pharmaceutical composition of mutated factor H binding proteins (fHbp) of A02, A42, A46, B44 and B107 of N. meningitides which is a vaccine.

In one of the embodiment, the invention also provides a method for raising an antibody response in a mammal, comprising administering a pharmaceutical composition of the invention to the mammal. The antibody response is preferably a protective and/or bactericidal antibody response.

In another embodiment, the invention also provides a method for protecting a mammal against N. meningitidis infection, comprising administering to the mammal a pharmaceutical composition of the invention.

In yet another embodiment, the mammal is preferably a human. The human may be an adult or, preferably, a child.

In one of the embodiment, the present invention provide a pharmaceutical composition of mutated factor H binding proteins (fHbp) which will generally be administered directly to a patient. Direct delivery may be accomplished by parenteral injection (e.g. Subcutaneously, intraperitoneally, intradermal, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml. The invention may be used to elicit systemic and/or mucosal immunity.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses (e.g. between 4-16 weeks), and between priming and boosting, can be routinely determined.

In one of the embodiment, the present invention provide a pharmaceutical composition of mutated factor H binding proteins (fHbp) comprise an immunologically effective amount of fHbp protein, as well as any other of other specified components, as needed. By immunologically effective amount, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to Synthesize antibodies, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The composition may be administered in conjunction with other immuno-regulatory agents.

### Examples

The present invention is further explained in the form of following examples. However, it is to be understood that the following examples are merely illustrative and are not to be taken as limitations upon the scope of the invention.

Example 1: Process of Preparation of Mutated Factor H Binding Proteins (fHbp) of A02 (Sequence ID 1), A42 (Sequence ID 2), A46 (Sequence ID 3), B44 (Sequence ID 4) and B107 (Sequence ID 5) of N. meningitidis Serogroup B

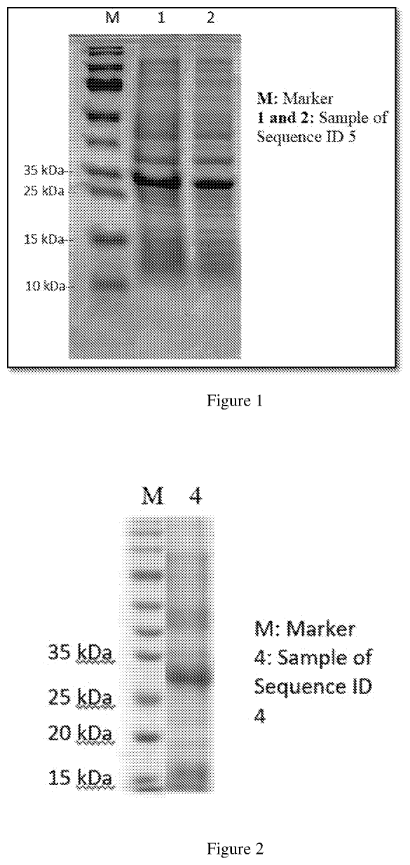

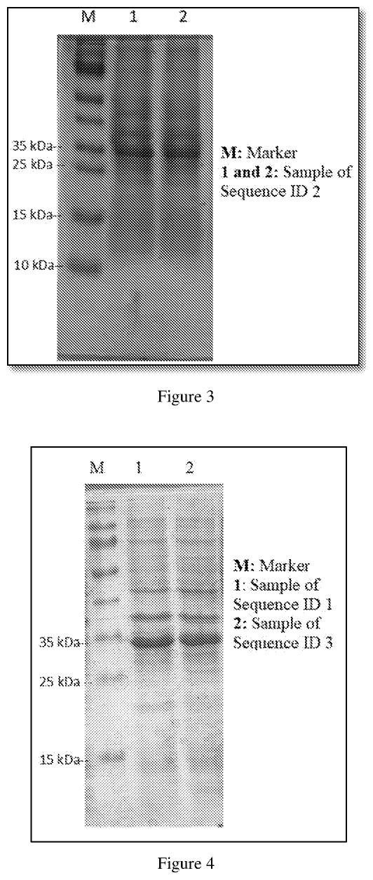

The cloning of the respective fHbp genes (having the P4 signal sequence at the start of N terminal) was done into expression vector pET303 (Thermo, USA). Further processes involved codon optimization, gene synthesis, sub cloning, transformation of E. coli DH5α (NEB, USA) strain, and the positive clone strain was extracted to obtain plasmid. All the five respective His-Tag [6 His-Tag] (at C terminal of) fHbp's were expressed in E. coli BL21 DE3 [NEB, USA] cells in LB media (Sigma, USA) having 100 μg/ml ampicillin. The induction for all the five fHbp's (of subfamily A and B) was performed (at an OD_{600 nm} between 0.6-0.8 using 1 mM IPTG (Sigma, USA) at 37° C., 250 rpm for 4 hours in the incubator shaker. Before induction, an uninduced sample was withdrawn and kept at the same conditions as described for induction. After induction, the total cell pellet was centrifuged at 6000 rpm for 20 mins and the pellet was stored and supernatant discarded. Both un-induced (not shown) as well as the induced fractions were analysed using SDS PAGE for the expression of respective fHbp proteins (FIG. 1-4). The induced fraction or the pellets were stored for further processing and purification of protein at −80° C.

Example 2: Purification of Mutated fhbp

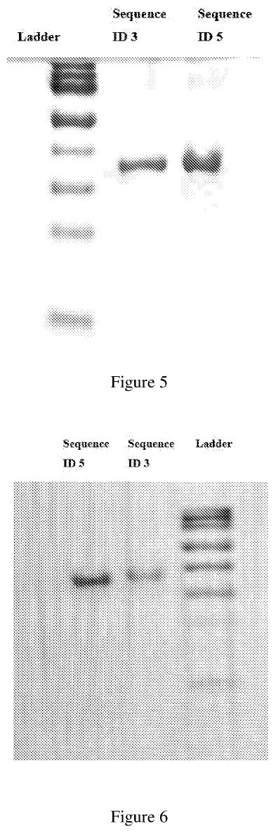

The mutated protein purification was carried out by Ni-NTA chromatography. Bacterial pellet of 1 L culture was resuspended in 50 ml Lysis buffer (20 mM Tris-Cl pH 8.0, 300 mM NaCl, 10 mM imidazole, 10% glycerol) containing Lysozyme, Protease inhibitor and Benzonase. The suspended pellets were incubated on ice for 30 min and sonicated for 3 min (30 sec pulse ON and 30 sec pulse OFF at 30% amplitude), and then centrifuged at 14000 rpm for 20 min at 10 C. The pellet was solubilized in 2.5% sarkosyl buffer (prepared in Lysis Buffer). The resuspended and solubilized pellets were kept at room temperature for 15 min and later centrifuged at 13000 rpm for 10 min at 10 C. Supernatant is harvested and diluted (approx. 9-10 times) in Lysis Buffer containing 1% NP 40 and proceeded for interaction with Ni-NTA resin. The column is washed with 5 resin bed volume of washing buffer No. 1 (20 mM Tris-Cl pH 8.0, 300 mM NaCl, 25 mM imidazole, 10% glycerol, 1% NP 40) and No. 2 (20 mM Tris-Cl pH 8.0, 500 mM NaCl, 25 mM imidazole, 10% glycerol, 1% NP 40). The bound His-tag proteins were eluted with 2 resin bed volume of Elution Buffer (20 mM Tris-Cl pH 8.0, 300 mM NaCl, 250 mM imidazole, 10% glycerol, 1% NP 40). The eluted fractions were analysed on SDS PAGE and relevant fractions were pooled. The purified fHbp proteins from both the subfamilies (A and B) were desalted and brought in phosphate buffer saline using PD 10 columns and analyzed using SDS PAGE and Western blot for the purified fHbp's (FIGS. 5 and 6).

### Example 2: Toxicity Study

Repeated Dose Subcutaneous Toxicity/Immunogenicity study with fHbp of the current invention in Swiss Albino Mice. The purpose of study was to assess the toxicity/immunogenicity when administered to mice via subcutaneous route at dose interval of 0 and 28 days.

In this study, the reference item was used at Low dose level. This will enable the comparison of toxic effects between test item and reference item.

This study provided information on the safety profile and potential toxic effects of fHbp of the current invention from repeated exposure (Day 0 and 28) over a prolonged period of time.
(a) Blood samples were drawn from the retro-orbital plexus using a glass capillary tube.
(b) Blood samples were centrifuged and serum was separated.
(c) Blood samples were drawn for immunogenicity analysis were collected from all the animals on day 0 (Pre-treatment), 14, 28 and 42 (Pre-termination). Blood samples were collected in Eppendorf tubes and centrifuged. After centrifugation serum was separated from the blood for immunogenicity analysis. Serum samples were stored at −20° C. until shipment. Serum samples were shipped to sponsor for immunogenicity analysis and immunogenicity analysis is performed and reported below in example 3.

All animals survived till the end of scheduled treatment period (Day 42), all animals were weighed, sacrificed and necropsied.

Conclusion: fHbp of the current invention were administered to Swiss Albino Mice on Day 0 and Day 28. No treatment related adverse effects observed in clinical signs, body weight, body weight change, feed consumption and macroscopic observation Hence, under the conditions of the experiment and based on the findings of the present study entitled “Repeated Dose Subcutaneous Toxicity/Immunogenicity study with fHbp of the current invention in Swiss Albino Mice”, “It is concluded that the fHbp of the current invention was safe and non-toxic to mice at the tested dose as mentioned in table 1

### Example 3: Immunogenicity Study

The composition comprising fHbp's of Neisseria meningitidis serogroup B composition were administered into animal models on Day 0 and Day 28. There were 8 groups that were made and then administered into animal model to check the effect of Subfamily A/B or both the subfamily together in a composition.

TABLE 1
GROUP DETAILS

| GROUPS (G) | COMPOSITION |
|---|---|
| G1 | G1 (Negative control) |
| G2 | G2 (Adjuvant control) |
| G3 | G3 (sequence ID No. 3 [12 μg] + Adjuvant) |
| G4 | G4 (sequence ID No. 5 [12 μg] + Adjuvant) |
| G5 | G5 (sequence ID No. 3 [12 μg] + sequence ID No. 5 |
| | [12 μg] + Adjuvant) |
| G6 | G6 (sequence ID No. 3 [12 μg] + sequence ID No. 5 |
| | [12 μg]) |
| G7 | G7 (sequence ID No. 3 [60 μg] + sequence ID No. 5 |
| | [60 μg]) |
| G8 | G8 (sequence ID No. 3 [60 μg] + sequence ID No. 5 |
| | [60 μg] + Adjuvant) |

### Procedure for Indirect ELISA:

The first and second dose was given at Day 0 and Day 28 respectively whereas the sera was collected at Day 0, Day 28 and Day 42.

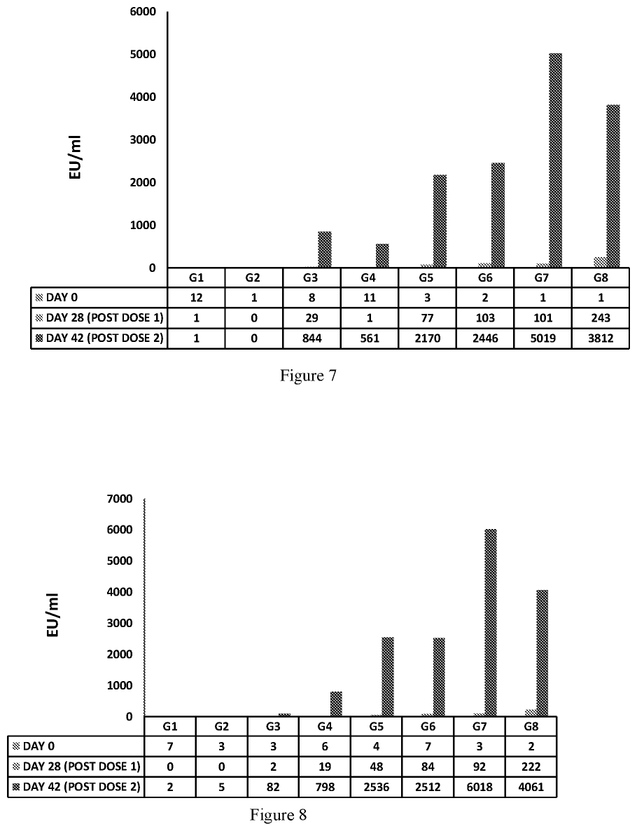

Indirect Enzyme linked Immuno Sorbent Assay (ELISA) was performed on pooled sera of the respective groups of Day 0, Day 28 and Day 42 as per Gheesling et al with slight modifications [Gheesling et al., J Clin Microbiol. 1994 June; 32(6): 1475-82.]. MaxiSorp 96 well plates were coated with 1 μg/ml of either fHbp of subfamily A or fHbp of subfamily B in 1×PBS buffer (pH 7.3±0.1) and each well was coated with 100 μl and having 100 ng of the antigen. After coating, the plates were kept at 2-8° C. at overnight incubation. The subsequent day, plates were washed with wash buffer (1×PBS buffer [pH 7.3±0.1]+0.1% tween 20 [v/v]). After the washing, blocking was done for 1 hour at room temperature {RT} [25±2° C.] with blocking buffer (1×PBS buffer [pH 7.3±0.1]+0.1% tween 20 v/v+5% fetal bovine serum [FBS]). Blocking was done with 200 μl of blocking buffer/well. In the time being, the pre dilutions of serum samples (1:800) and the quality control (QC) serum (1:1600) were prepared using assay. The ELISA performed for both the fHbp's of subfamily A or subfamily B were having the same pre-dilutions for the serum samples as well as the QC serum [QC serum was made in house, utilizing the groups 7 and 8 of Day 42. Equal quantity of serum was pooled from each mice of both the groups (7 and 8) of Day 42 to prepare the QC serum]. After blocking, the plates were washed and 100 μl/well of assay buffer (1×PBS buffer [pH 7.3±0.1]+0.1% tween 20 v/v+5% FBS) was put in the coated plates from row B till row H whereas 200 μl/well of QC serum (in duplicate) and serum samples (in duplicate) respectively was put in row A. Subsequently six two-fold serial dilutions were performed until row G in the corresponding wells. All the wells of row H had only assay buffer and were designated as blank as it was devoid of serum. The plates were put for 2 hours of incubation time at RT. After incubation, washing was performed using wash buffer and the plates were incubated for 1 hour at RT with secondary antibody (anti mouse conjugated with horseradish peroxidase (HRP). It was 100 μl/well of secondary antibodies and the dilution was 1:16000 for subfamily A fHbp coated plates whereas the dilution was 1:8000 for subfamily B fHbp coated. The preparation of the secondary antibody dilutions were made in the assay buffer. After the incubation was complete, the plates were washed and the TMB substrate (3,3′,5,5′-Tetramethylbenzidine) was added as 100 μl/well for 10 minutes (mins) at RT. After 10 mins, 50 μl/well of 2M H₂SO₄ was added to end the reaction and the absorbance of the respective plates was read at 450 nm/620 nm. The QC serum prepared was given an arbitrary value of 5000 ELISA Units/ml or EU/ml and used as a standard in each plate. The standard of each plate was utilized to generate a standard ELISA curve facilitating in the estimation of IgG concentrations of the corresponding samples/groups using the combistats software (FIGS. 7 and 8).

Conclusion: The study conducted indicates the immunogenic potential of both fHbp A and fHbp B of the current invention post two doses (Day 42) as described in FIGS. 7 and 8. The IgG concentrations in EU/ml are higher in group 7 which is having higher dose combination of 60 μg each of fHbp A and fHbp B compared to group 5 where the dose is 12 μg each of fHbp A and fHbp B. Moreover it is observed that combination groups having both the fHbp's (subfamily A and B) are also having higher IgG's compared to the groups having fHbp from one subfamily i.e. either subfamily A or subfamily B.

SEQUENCE LISTING

| | | SE- |
|---|---|---|
| | | QUENCE |
| NO. | AMINO ACID SEQUENCE | ID NO. |
| Mutated | CSSGGGGSGGGVVAGGAAVTLADALTAPL | 1 |
| protein | DHKDKGLKSLTLEDSIPQNGTLTLSAQGAE | |
| A02 | KTFKAGDKDNSLNTGKLKNDKISRFDFVQ | |
| | KIEVDGQTITLASGEFQIYKQDHSAVVALQI | |
| | EKINNPDKIDSLINQRSFLVSEAAVVRTAFN | |
| | QLPGGKAEYHGKAFSSDDPNGRLHYSIDFT | |
| | KKQGYGGIEHLKTPEQNVELASAELKADE | |
| | KSHAVILGDTRYGSEEKGTYHLALFGDRA | |
| | QEIAGSATVKIGEKVHEIGIAGKQ | |
| Mutated | CSSGGGGVAAGVVAGGAAVTTAPLDHKD | 2 |
| protein | KGLKSLTLEDSISQNGTLTLSAQGAERTFK | |
| A42 | AGNKDNSLNTGKLKNDKISRFDFVQKIEVD | |
| | GQTITLASGEFQIYKQNHSAVVALQIEKINN | |
| | PDKIDSLINQRSFLVSSLGGSEAAVVRLPGG | |
| | KAEYHGKAFSSDDPNGRLHYSIDFTKKQG | |
| | YGRIEHLKTPEQNVELAAAELKADEKSHA | |
| | VILGDTRYGSEEKGTYHLALFGDRAQEIAG | |
| | SATVKIGEKVHEIGIAGKQ | |
| Mutated | CSSGGGGSGSGVVAGGAAVTLADALTTPL | 3 |
| protein | DHKDKGLKSLTLEDSIPQNGTLTLSAQGAE | |
| A46 | KTFKAGDKDNSLNTGKLKNDKISRFDFVQ | |
| | KIEVDGQTITLASGEFQIYKQDHSAVVALQI | |
| | EKINNPDKIDSLINQRSFLVSEAAVVRTAFN | |
| | QLPGGKAEYHGKAFSSDDAGGKLTYTIDF | |
| | ASKQGHGKIEHLKTPEQNVELAAAELKAD | |
| | EKSHAVILGDTRYGSEEKGTYHLALFGDR | |
| | AQEIAGSATVKIGEKVHEIGIAGKQ | |
| Mutated | CSSGGGGSGGGVVAGGAAVTLADALTAPL | 4 |
| protein | DHKDKGLKSLTLEDSISQNGTLTLSAQGAE | |
| B44 | RTFKAGDKDNSLNTGKLKNDKISRFDFIRQ | |
| | IEVDGQLITLESGEFQVYKQSHSALTALQTE | |
| | QVQDSEHSGKMVAKRQFRISEAAVVRTSF | |
| | GKLPKDVMATYRGTAFGSDDAGGKLTYTI | |
| | DFAAKQGHGKIEHLKSPELNVDLAAADIKP | |
| | DEKHHAVISGSVLYNQAEKGSYSLGIFGGQ | |
| | AQEVAGSAEVETANGIRHIGLAAKQ | |
| Mutated | CSSGGGGVAAGVVAGGAAVTTAPLDHKD | 5 |
| protein | KGLKSLTLEDSISQNGTLTLSAQGAERTFK | |
| B107 | AGDKDNSLNTGKLKNDKISRFDFIRQIEVD | |
| | GQLITLESGEFQVYKQSHSALTALQTEQVQ | |
| | DSEHSGKMVAKRQFRIGDIVGSEAAVVRLP | |
| | KDVMATYRGTAFGSDDAGGKLTYTIDFAA | |
| | KQGHGKIEHLKSPELNVDLAAADIKPDEKH | |
| | HAVISGSVLYNQAEKGSYSLGIFGGQAQEV | |
| | AGSAEVETANGIRHIGLAAKQ | |

Although the preferred embodiments of the present invention and their respective variations have been described, people having ordinary skills in the art would envision various modifications of those embodiments.

Accordingly, the present invention should not be limited to precise forms and manners in the above disclosure and description but should simply be taken by way of examples. Thus, the present invention can be varied and modified without departing the true scope and spirit thereof as defined in the appended claims.

### Advantages of the Present Invention

The present invention provides mutated factor H binding proteins (fHbp) of A02, A42, A46, B44 and B107 of N. meningitidis serogroup B.

The present invention provides an immunological composition as described herein above comprises of combination of mutated subfamily A and subfamily B protein hence provide broader coverage and useful for complete prophylaxis against meningococcal diseases caused by pathogenic N. meningitidis.

The present invention provides an immunological composition as described herein above for complete prophylaxis against meningococcal diseases caused by pathogenic N. meningitidis.

The present invention provides a novel, specific and industrially advantageous mutated factor H binding proteins (fHbp) of A02, A42, A46, B44 and B107 of N. meningitidis serogroup B that are capable of complete prophylaxis against meningococcal diseases. These compositions shall be fulfilling the unmet medical need for Meningitis prophylaxis and treatment.

1. A mutated factor H binding protein (fHbp) of A02, A42, A46, B44 and B107 of N. meningitidis family.

2. The mutated fHbp of claim 1, wherein the amino acid sequence of A02, A42, A46, B44 and B107 has at least 90% sequence identity to SEQ ID NOs 1, 2, 3, 4 and 5, respectively.

3. A process of preparation of mutated factor H binding proteins (fHbp) as claimed in claim 1.

4. A process of preparation of mutated factor H binding proteins (fHbp) as claimed in claim 1, wherein the process is selected from recombinant process, synthetic process or combination of both.

5. A process of preparation of mutated factor H binding proteins (fHbp) of A02, A42, A46, B44 and B107 of N. meningitidis serogroup B fHbp comprising steps of:
a) Cloning of the individual fHbp gene with the signal sequence into expression vector,
b) Plasmid extraction,
c) Transformation
d) Induction of the respective fHbp's
e) Purification of respective fHbp's

6. A pharmaceutical composition comprising at least two of the following:
a) mutated protein of fHbp family A02 of N. meningitidis (protein of sequence ID. 1);
b) mutated protein of fHbp family A42 of N. meningitidis (protein of sequence ID. 2);
c) mutated protein of fHbp family A46 of N. meningitidis (protein of sequence ID. 3);
d) mutated protein of fHbp family B44 of N. meningitidis (protein of sequence ID. 4); and
e) mutated protein of fHbp family B107 of N. meningitidis (protein of sequence ID. 5).

7. The composition as claimed in claim 5, useful for the treatment or prevention or diagnosis of N. meningitidis disease caused by MenA, MenB, MenC, MenW, MenY, and MenX serogroups of N. meningitides.

8. The composition as claimed in claim 5, useful for the treatment or prevention or diagnosis of N. meningitidis disease caused by MenB.

9. The composition as claimed in claim 5, further comprise of a pharmaceutically acceptable carrier, diluent or adjuvant.

10. The composition as claimed in claim 8, wherein the pharmaceutically acceptable carrier is selected from but not limited to, buffer, antioxidant, preservative, isotonic agent and chelating agent.

11. The composition as claimed in claim 5, wherein the composition is fully liquid and/or lyophilized modified fHbp from subfamily A and/or modified fHbp from subfamily B of N. meningitidis serogroup B and/or mixed with fully liquid and/or lyophilized conjugated capsular polysaccharides from N. meningitidis serogroup A, C, W135, X and/or Y.

12. The composition as claimed in claim 5, wherein the composition is a Vaccine.

13. The composition as claimed in claim 5, wherein the composition is administered Subcutaneously, intraperitoneally, intradermal, intravenously, intramuscularly, or to the interstitial space of a tissue), or by rectal, oral, vaginal, topical, transdermal, intranasal, ocular, aural, pulmonary or other mucosal route.

14. A process of preparation of mutated factor H binding proteins (fHbp) as claimed in claim 2.

15. A process of preparation of mutated factor H binding proteins (fHbp) as claimed in claim 2, wherein the process is selected from recombinant process, synthetic process or combination of both.


SEQUENCE LISTING (5 sequences)

SEQ ID NO: 1 - protein, 262 aa, Homo sapiens
CSSGGGGSGGGVVAGGAAVTLADALTAPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSEAAVVRTAFNQLPGGKAEYHGKAFSSDDPNGRLHYSIDFTKKQGYGGIEHLKTPEQNVELASAELKADEKSHAVILGDTRYGSEEKGTYHLALFGDRAQEIAGSATVKIGEKVHEIGIAGKQ

SEQ ID NO: 2 - protein, 257 aa, Homo sapiens
CSSGGGGVAAGVVAGGAAVTTAPLDHKDKGLKSLTLEDSISQNGTLTLSAQGAERTFKAGNKDNSLNTGKLKNDKISRFDFVQKIEVDGQTITLASGEFQIYKQNHSAVVALQIEKINNPDKIDSLINQRSFLVSSLGGSEAAVVRLPGGKAEYHGKAFSSDDPNGRLHYSIDFTKKQGYGRIEHLKTPEQNVELAAAELKADEKSHAVILGDTRYGSEEKGTYHLALFGDRAQEIAGSATVKIGEKVHEIGIAGKQ

SEQ ID NO: 3 - protein, 262 aa, Homo sapiens
CSSGGGGSGSGVVAGGAAVTLADALTTPLDHKDKGLKSLTLEDSIPQNGTLTLSAQGAEKTFKAGDKDNSLNTGKLKNDKISRFDFVQKIEVDGQTITLASGEFQIYKQDHSAVVALQIEKINNPDKIDSLINQRSFLVSEAAVVRTAFNQLPGGKAEYHGKAFSSDDAGGKLTYTIDFASKQGHGKIEHLKTPEQNVELAAAELKADEKSHAVILGDTRYGSEEKGTYHLALFGDRAQEIAGSATVKIGEKVHEIGIAGKQ

SEQ ID NO: 4 - protein, 263 aa, Homo sapiens
CSSGGGGSGGGVVAGGAAVTLADALTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRISEAAVVRTSFGKLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ

SEQ ID NO: 5 - protein, 258 aa, Homo sapiens
CSSGGGGVAAGVVAGGAAVTTAPLDHKDKGLKSLTLEDSISQNGTLTLSAQGAERTFKAGDKDNSLNTGKLKNDKISRFDFIRQIEVDGQLITLESGEFQVYKQSHSALTALQTEQVQDSEHSGKMVAKRQFRIGDIVGSEAAVVRLPKDVMATYRGTAFGSDDAGGKLTYTIDFAAKQGHGKIEHLKSPELNVDLAAADIKPDEKHHAVISGSVLYNQAEKGSYSLGIFGGQAQEVAGSAEVETANGIRHIGLAAKQ